(19) United States
(12) Patent Application Publication
(10) Pub. No.: US 2007/0135732 A1
(43) Pub. Date: Jun. 14, 2007

(54) Flexible mandril

(75) Inventors: Christopher G. Dixon, Bloomington, IN (US); James M. Carlson, Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/605,019

(22) Filed: Nov. 28, 2006

Related U.S. Application Data
(60) Provisional application No. 60/749,829, filed on Dec. 13, 2005.

(51) Int. Cl. A61B 5/00

(52) U.S. Cl. 600/583

(57) ABSTRACT

A support member for providing core support to an elongated medical device. The support member comprises a generally cylindrical substrate having a proximal end and a distal end, and having a mass such that the respective proximal and distal ends are visible under medical imagery. The substrate is sized to be received in a lumen of the medical device, and has a plurality of grooves, axially disposed along the length of the substrate. The support member may have a greater number of grooves per unit length of the substrate at the distal end than at the proximal end, thereby imparting an increased flexibility to the distal end when compared to the proximal end.

### RELATED APPLICATION

The present patent document claims the benefit of the filing date under 35 U.S.C. §119(e) of Provisional U.S. Patent Application Ser. No. 60/749,829, filed Dec. 13, 2005, which is hereby incorporated by reference.

### BACKGROUND

1. Technical Field

The present invention relates to a medical device, and more particularly, to a support device, such as a mandril, for providing core support for a wire guide, catheter, and the like.

2. Background Information

Modern medical practice often involves the percutaneous insertion of medical devices into body vessels for transport to target sites deep within the vasculature. During transport, a device must often negotiate very sharp bends, and traverse otherwise tortuous passageways to arrive at the desired site. The device must therefore be structured to accommodate such bends and passageways. In addition, considerable skill is required on the part of the physician to direct the medical device to the appropriate site.

In order to traverse these bends and tortuous passageways, a device must be sufficiently flexible to make the required turns. At the same time, the device must be sufficiently rigid to resist kinking, and to transmit a sufficient amount of torque to permit continuous advancement of the device in the vessel. Numerous prior art devices have been developed in attempts to address either, or both, of these competing interests of flexibility and rigidity.

In order to enhance the rigidity of certain medical devices, such as wires guides, catheters, and the like, to permit passage of the devices through the vasculature, a core support structure is sometimes inserted into the lumen of the device. The core support structure, such as a mandril, adds strength to the device, thereby enabling the device to pass through vessels that might otherwise be difficult, if not impossible, to traverse. On some occasions, a solid mandril is inserted that occupies virtually the entire lumen of the medical device. However, when a solid mandril is inserted, the distal end of the medical device will often have insufficient flexibility to negotiate tight angles, or to otherwise traverse the tortuous passages. In order to avoid this occurrence, the clinician may instead insert the solid mandril only partway through the lumen. In this way, the proximal portion of the device through which the mandril has been inserted may achieve the desired strength for insertion into the vessel, but the unsupported distal portion may lack sufficient strength to resist bending or kinking.

Other attempts have been made to optimize these competing conditions of flexibility and rigidity. In one such attempt, a highly tapered mandril has been inserted into the lumen of the medical device. The mandril gradually tapers from a larger diameter proximal end to a much smaller diameter distal end. When inserted into the lumen of the medical device, the larger diameter end of the tapered mandrel adds rigidity and support to the proximal end of the device. The smaller diameter end of the tapered mandril provides less rigidity to the distal end, and thereby enables this end to retain a greater degree of flexibility. Although this device provides a gradual transition from the desired rigidity of the proximal end to the desired flexibility of the distal end, the mandril is tapered to an extent that only a limited amount of mass remains at the distal end. As a result, the distal portion of the mandril may not be sufficiently radiopaque to be visible under medical imaging diagnostic procedures, such as x-ray fluoroscopy. In view of the increased use of imaging techniques in modern medical practice, this lack of visibility may eliminate an otherwise important tool for the physician when tapered mandrils are utilized.

Another attempt to optimize the competing conditions of flexibility and rigidity in a medical device involved the use of a tubular mandril having a spiral cut-out along the length of the tube. The tubular mandril is typically laser cut along its length such that the turns of the spiral cut become gradually narrower from the proximal end of the tube to the distal end, and/or the spacing between the turns gradually increases from the proximal end to the distal end. As a result, the tubular mandril support is more rigid at the proximal end, and more flexible at the distal end. However, since the tubular structure has a hollow interior, it may have insufficient mass to be visible under medical imaging diagnostic equipment.

It would be desirable to provide a core support structure for a medical device that exhibits sufficient flexibility to enable the device to traverse tortuous passageways, and yet retains sufficient strength to resist kinking, and to transmit sufficient torque to assist in the advancement of the device in the vessel. In addition, it would be desirable to provide such a core support structure that retains sufficient radiopacity to be visible under medical imagery.

### BRIEF SUMMARY

The problems of the prior art are addressed by the present invention. In one form thereof, the present invention relates to a support member for providing core support to a medical device. The support member comprises a generally cylindrical substrate having a proximal end and a distal end. The substrate is sized to be received in a lumen of the medical device, and has a plurality of cut-out portions, such as a plurality of grooves, axially disposed along the length of the substrate. The support member may have a greater number of grooves per unit length of the substrate at the distal end than at the proximal end, thereby imparting an increased flexibility to the distal end when compared to the proximal end.

In another form thereof, the invention comprises a method of making a support member for an elongated medical device. A generally cylindrical substrate having a proximal end and a distal end is provided, wherein the substrate is sized to be received in a lumen of the elongated medical device. The proximal and distal ends of the substrate are visible under medical imagery. A plurality of cut-out portions, such as grooves, are formed along a length of the substrate by means such as laser cutting. The cut-out portions are sized and arranged such that the flexibility of the distal end of the substrate distal exceeds the flexibility of the substrate proximal end.

### BRIEF DESCRIPTION OF THE DRAWINGS

FIG. 1 is side view of a flexible mandril according to an embodiment of the present invention;

FIG. 2 is a cross-sectional view of the flexible mandril of FIG. 1 positioned within the lumen of a catheter;

FIG. 3 is a side view of another embodiment of a flexible mandril according to the present invention; and

FIGS. 4-10 illustrate further embodiments of a flexible mandril according to the present invention.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive mandril, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient.

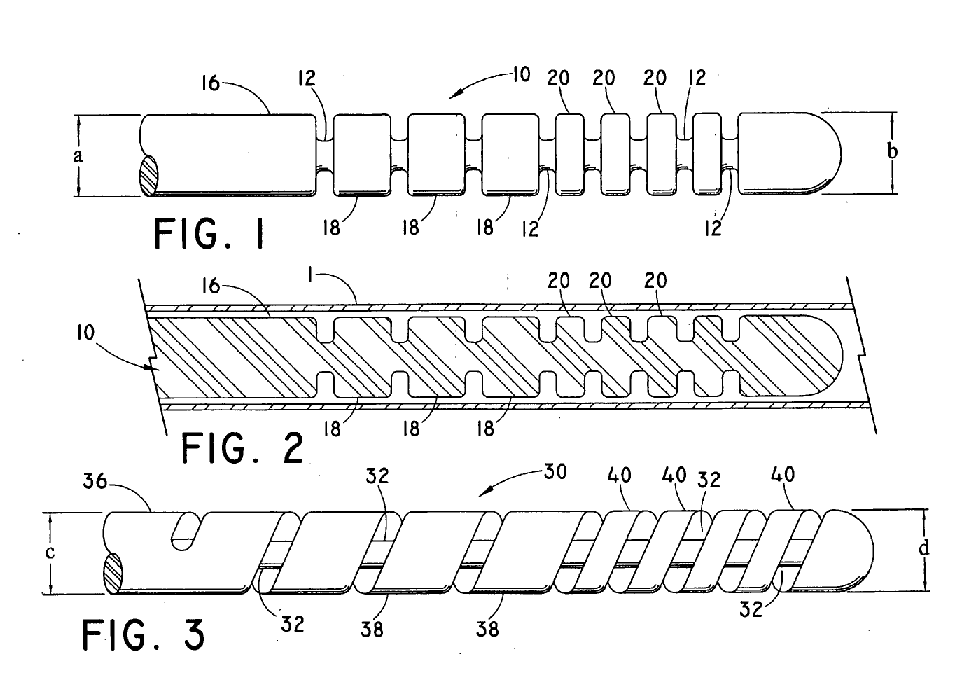

FIG. 1 illustrates a side view of a flexible mandril 10 according to an embodiment of the present invention. Mandril 10 is sized for insertion into a conventional medical device, such as a catheter, wire guide, and the like, to provide support for the medical device during insertion of the device into the vasculature of a patient. Following withdrawal of the mandril, the inserted medical device can then be used in well-known manner for, e.g., the insertion of an interventional device, such as a stent, stent-graft, etc., or as a conduit for the introduction of a liquid medicine into the vessel, and/or the aspiration of a fluid from the vessel.

In the embodiment shown in FIG. 1, mandril 10 has a substantially constant outer diameter. By “substantially constant outer diameter” is meant that the diameter a may be the same as diameter b, but that minor variations are permissible between diameter a and diameter b. If any such variations are present, the respective lengths of diameter a and diameter b will preferably not vary from each other by more than about 20%, more preferably by not more than about 10%, and most preferably by not more than about 1%. Thus, for example, in the most preferred arrangement, the opposite axial ends of a 0.010 inch (0.25 mm) wire would not vary from each other by more than about +/−0.0001 inch (0.0025 mm). As a result, mandril 10 provides dimensional support for the vessel virtually throughout the entire length of the mandril. Mandril 10 can be formed to have virtually any length, and diameter, consistent with the nature of the medical device in which it is to be received, and the ultimate medical procedure in which it is to be utilized. Those skilled in the art are readily capable of determining an appropriate length and diameter of mandril 10 for use in a particular medical procedure.

As illustrated in the embodiment of FIG. 1, mandril 10 comprises a substantially solid cylindrical structure having a plurality of grooves 12 formed therein. In this embodiment, grooves 12 comprise generally circumferential cut-out portions axially disposed along the length of mandril 10. Grooves 12 can be spaced at any desired position along the length of mandril 10. However, in most cases it will be desirable for the mandril to exhibit increased flexibility at the distal end as compared to the proximal end. In this instance, grooves 12 will be cut into the otherwise solid mandril such that there are more grooves per unit of mandril length at the distal end than at the proximal end. This arrangement is shown in FIG. 1. The resulting proximal mandril sections 16 are of much greater length than the distal mandril sections 20. Stated another way, the mandril has less mass at the distal end than at the proximal end. As a result of this structure, the proximal section will exhibit a greater degree of strength and rigidity than the distal section, while the distal section will exhibit a greater degree of flexibility than the proximal section.

In order to smooth the transition from the more rigid proximal section to the more flexible distal section, additional grooves can be provided to define intermediate mandril sections 18. The number of grooves per unit length of the mandril at this intermediate portion will be intermediate the numbers at the respective proximal and distal end. As a result, intermediate sections 18 have a length intermediate the respective lengths of the proximal mandril sections 16 and distal mandril sections 20. This optional arrangement provides for increased strength and rigidity at the proximal end, increased flexibility at the distal end, and a gradual transition between the rigid proximal end and the flexible distal end.

Although FIG. 1 illustrates three discrete mandril sections, namely the proximal section, intermediate section and distal section, those skilled in the art will appreciate that more, or fewer discrete sections may be provided. For example, even though the proximal section will normally be the section of greatest rigidity, and the distal section will normally be the section of greatest flexibility, any number of transition sections can be provided between the proximal and distal sections. Similarly, although grooves 12 have been shown and described as extending around the entire circumference of mandril 10, those skilled in the art will appreciate that this need not always be so. Rather, each groove 12 can extend around all, or only a segment, of the circumference of the mandril.

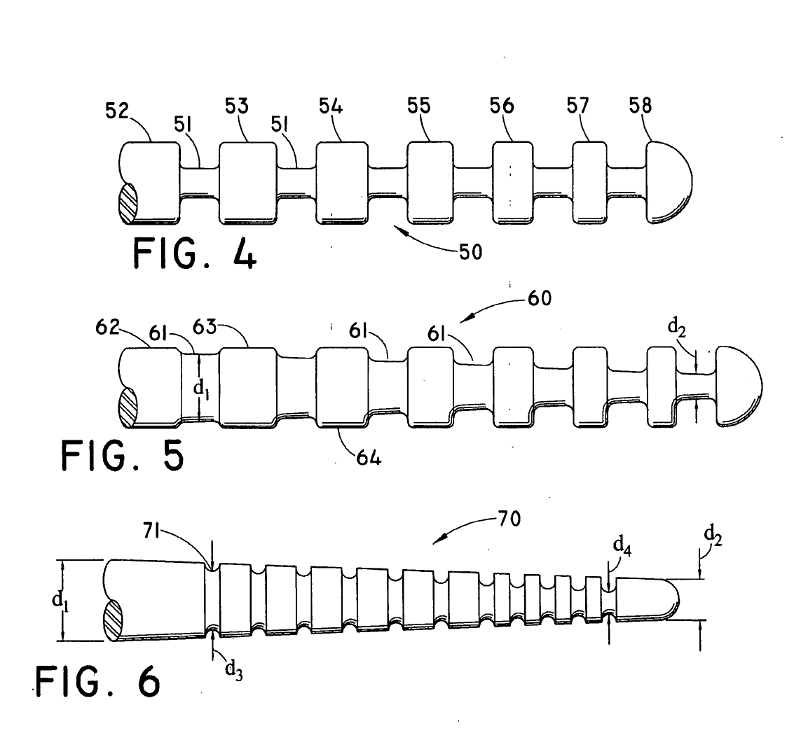

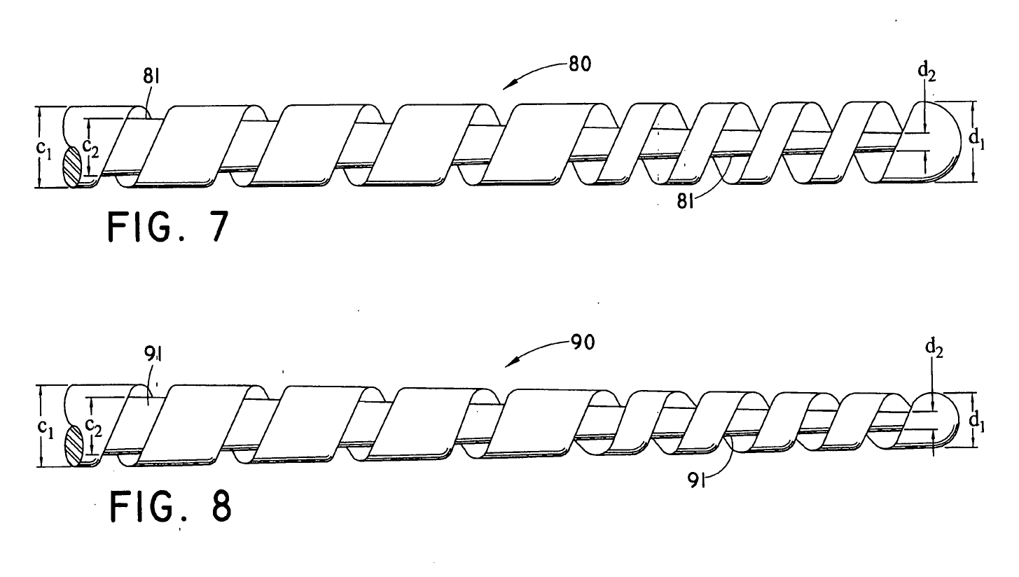

FIG. 2 illustrates a cross-sectional view of flexible mandril 10 of FIG. 1 positioned within the lumen of a medical device, such as a catheter 1. Although mandril 10 is illustrated in the figure, those skilled in the art will appreciate that any of the mandril configurations described hereinafter, and illustrated in FIGS. 3-10 can be substituted. As stated, in order to provide optimal support to the catheter, it is preferred that the mandril have a diameter such that the mandril will substantially occupy the internal diameter of the catheter. Additionally, it is preferred that the mandril will have a diameter such that sufficient mass exists at each distal end to render the device visible under medical imagery.

FIG. 3 illustrates another embodiment of the invention, illustrating a mandril 30 having spiral cut grooves 32. Spiral cut mandril 30 is otherwise generally similar to mandril 10 of FIG. 1, with the exception of the grooves 32 cut in a generally spiral pattern. In this embodiment, mandril 30 also has a substantially constant diameter, that is, diameter c may be the same as diameter d, but that minor variations are permissible between diameter c and diameter d. As with the embodiment of FIG. 1, mandril sections 36 in the proximal end are of greater length than the mandril sections 40 at the distal end. Sections 38 are provided intermediate the proximal sections 36 and distal sections 40. This arrangement provides increased strength and rigidity, and the concomitant reduced flexibility, at the proximal end compared to the distal end.

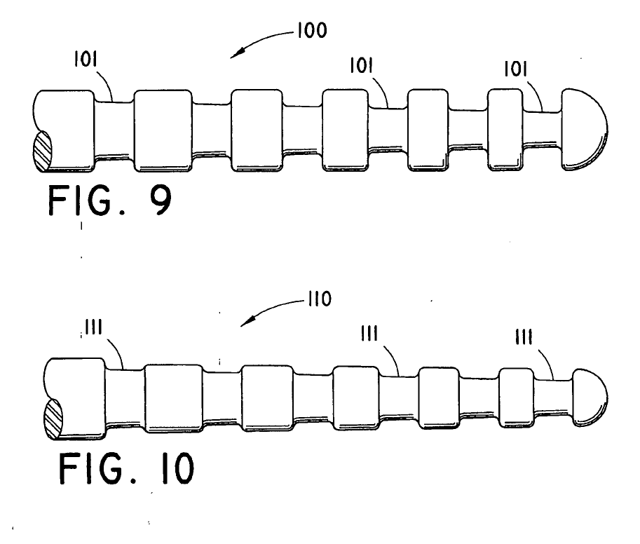

In the embodiments of FIGS. 1 and 3, each of the proximal section(s) 16, 36 intermediate sections 18, 38 and distal sections 20, 40 have the same length as the other sections within the respective category. In other words, each distal section 20 has the same length as other distal sections 20, each intermediate section 18 has the same length as other intermediate sections 18, etc. However, this need not necessarily be the case. FIG. 4 illustrates an embodiment wherein grooves are cut into the mandril in a manner such that the distance between successive grooves 51 decreases in a gradual and continuous manner from the proximal end to the distal end of mandril 50. As a result, the length of respective mandril sections 52, 53, 54, etc., decrease in a gradual and continuous fashion from the proximal end of mandril 50 to the distal end. In this manner, the rigidity of the mandril gradually decreases from the proximal end of the mandril to the distal end, while the flexibility gradually increases.

There are numerous other ways in which the flexibility along the length of the mandril may be controlled, and yet the mandril will retain sufficient overall mass to be visible under medical imaging techniques. For example, flexibility may be controlled by varying the depth of the grooves. One example of this arrangement is shown in FIG. 5. In this embodiment, mandril 60 includes mandril sections 62, 63, 64, etc., separated by respective grooves 61. Grooves 61 vary in depth from the proximal end, wherein at least one of the grooves 61 is cut to a minimum depth d₁, to the distal end, wherein at least one of the grooves is cut to the maximum depth d₂. Preferably, the respective depth of successive grooves increases in a gradual and continuous manner from the proximal end of the mandril to the distal end. Those skilled in the art will appreciate that, in most instances, a deeper groove will provide greater flexibility to a mandril section than a shallower groove.

Another alternative embodiment is shown in FIG. 6. In this embodiment, the mandril may, but need not, have a substantially constant diameter. In the embodiment illustrated, the mandril 70 is tapered from the proximal end to the distal end. This arrangement differs from the previous embodiments in that the proximal diameter d₁ is greater than the distal diameter d₂. However, unlike prior art tapered mandrils, the distal end does not taper to a sharp point. Rather, the distal end retains sufficient mass to be visible under conventional imaging techniques. Although the taper provides a certain element of enhanced flexibility at the distal end when compared to the proximal end, distal flexibility may optionally be even further enhanced by providing grooves along the length of the mandril. In the embodiment shown, grooves 71 increase in depth from the proximal end to the distal end, such that d₃>d₄. As a still further means of controlling the flexibility of the mandril, the above-described techniques can be combined with still other options, such as in this case, varying the spacing between the grooves such that the distance between adjacent grooves is less at the distal end of the mandril than at the proximal end.

Another alternative embodiment is shown in FIG. 7. In this embodiment, grooves 81 are spiral cut into mandril 80. The diameter c₁ at the proximal end of the mandril is substantially equal to the diameter d₁ at the distal end. Grooves 81 extend deeper into mandril 80 at the distal end than at the proximal end, or in other words, diameter c₂>d₂.

Another alternative embodiment is shown in FIG. 8. In this variation, grooves 91 are spiral cut into mandril 90. Proximal diameter c₁ of mandril 90 is greater than distal diameter d₁. The flexibility of the mandril further increases toward the distal end by increasing the depth of the cut into the mandril from the proximal end to the distal end, such that c₂>d₂.

FIGS. 9 and 10 illustrate still further embodiments. In FIG. 9, the depth of respective grooves 101 of mandril 100 increases from the proximal end of mandril 100 to the distal end. In this embodiment, flexibility is further enhanced by decreasing the distance between respective grooves from the proximal end to the distal end. In FIG. 10, the outside diameter of mandril 110 decreases from the proximal end to the distal end, and the distance between successive grooves 111 decreases. Each of these factors contributes to the enhanced flexibility at the distal end compared to the proximal end.

Those skilled in the art will thus appreciate that when the teachings of the present invention are followed, there is virtually no limit to the number of ways that a mandril can be altered to adjust its flexibility. As a result, the medical device in which the mandril is received can effectively traverse tortuous passageways, retain sufficient strength to resist kinking, transmit sufficient torque to assist in the advancement of the device in the vessel, and retain sufficient radiopacity to be visible under medical imagery. A particular mandril can be provided with any combination of the features described herein. Those skilled in the art can readily select a particular feature for enhancing flexibility, or a combination of features, based upon the particular medical procedure with which the mandril is to be used, and in further view of the requirements of the imaging technique that is to be utilized.

Although the invention has largely been described and shown herein with grooves cut into the mandril, the invention is not so limited. Rather, other cut-out configurations can be made along the length of the mandril. For example, rather than including grooves cut circumferentially around the body of the mandril, the same or a similar effect can be achieved by providing notches or like cut-out portions along the body of the mandril. The respective cut-outs can be provided at desired depths, relative spacings, configurations, etc., to achieve the desired increase in flexibility from the proximal end to the distal end, and to retain sufficient mass to be visible under medical imagery. Thus, the invention includes any type, and combination, of cut-out (such as a groove), of any dimension, that may be cut or otherwise formed along the length of the mandril.

The grooves or other cut-outs can be formed in the mandril by any conventional means for forming grooves or other shapes in a substrate. One particularly preferred method of forming cut-outs, such as grooves, in such small diameter substrates is by laser cutting the grooves into the solid mandril. Laser cutting enables an operator to closely control all facets of the formation of the grooves, such as depth, width, spacing, etc. In some instances other well-known methods of forming such grooves, such as chemical etching, machining, etc., may be substituted. Those skilled in the art can readily determine an appropriate manner for forming such cut-outs in view of the teachings of this invention. In most cases, it is envisioned that the mandril will have a diameter of about 0.011 to about 0.038 inch (0.28 to 0.97 mm), and, as explained, in some embodiments the diameter may vary between the proximal and distal ends. Generally, the grooves or other cut-outs will extend radially inwardly into the mandril about 0.003 to 0.015 inch (0.08 to 0.38 mm).

The flexible mandril described herein can be made of any conventional medical grade material that has sufficient strength to provide support in those areas, such as the proximal end, wherein support is a prime concern, and that is capable of exhibiting sufficient flexibility in those areas, such as the distal end, wherein flexibility is a prime concern. Non-limiting examples of such materials include stainless steel (e.g., stainless steel type 304V), various shape memory and superelastic materials such as nitinol, cobalt or nickel chromium based superalloys, precious metals and alloys, refractory metals and alloys, various polymeric materials, and composites. Those skilled in the art can readily identify an appropriate material in view of the particular use for which the flexible mandril is to be employed.

During formation of the mandril, it may be advisable to incorporate some detailing, such as electropolishing, to round off any sharp corners that may be present following centerless grinding. Another alternative is to extrude a jacket formed of a suitable polymer, such as polyurethane, over the centerless ground area. The polyurethane jacket offers flexibility, and also provides protection to the arteries against sharp corners. The use of a jacket also offers the advantage that it can be hydrophilically coated to improve trackability of the mandril in the body. Other known techniques for reducing sharp edges, such as bead or grit blasting, may also be used in an appropriate case. Those skilled in the art are capable of incorporating routine details into the formation of such mandrils such that the mandril is useful for a particular purpose.

As stated, a feature of the present invention is that the mandril retains sufficient mass, even at its distal end, such that is visible under modern medical imaging techniques. Thus, even the distal portion of the mandril is sufficiently radiopaque to be visible under imaging techniques, such as x-ray fluoroscopy. Other medical imaging techniques that can benefit from the teachings of the present invention include, but are not necessarily limited, to medical resonance imaging (MRI) and computer tomography (CT).

It is therefore intended that the foregoing detailed description be regarded as illustrative rather than limiting, and that it be understood that it is the following claims, including all equivalents, that are intended to define the spirit and scope of this invention.

1. A support member for providing core support to a medical device, said support member comprising:
a generally cylindrical substrate having a proximal end and a distal end, said substrate sized to be received in a lumen of said medical device, said substrate having a plurality of cut-out portions axially disposed along a length thereof.

2. The support member of claim 1, wherein said substrate has sufficient mass such that said proximal end and said distal end are visible under medical imaging.

3. The support member of claim 1, wherein said substrate has a substantially constant diameter from said proximal end to said distal end.

4. The support member of claim 2, wherein said cut-out portions comprise a plurality of grooves.

5. The support member of claim 4, wherein said grooves are disposed at at least the distal end of said substrate.

6. The support member of claim 5, wherein said substrate has a greater number of grooves per unit length of said substrate at said distal end than at said proximal end.

7. The support member of claim 5, wherein said grooves are spaced to define a proximal section having a smaller number of grooves per unit of substrate length and a distal section having a greater number of grooves per unit of substrate length.

8. The support member of claim 7, further comprising an intermediate section having a larger number of grooves per unit of substrate length than said proximal section, and a smaller number of grooves per unit of substrate length than said distal section.

9. The support member of claim 4, wherein said grooves comprise respective rings disposed around a circumferential surface of said substrate.

10. The support member of claim 4, wherein said grooves comprise a spiral pattern formed along the circumference of said substrate.

11. The support member of claim 1, wherein said support member is formed from a member selected from the group consisting of stainless steel, shape memory materials, cobalt or nickel chromium based superalloys, precious metals and alloys thereof, refractory metals and alloys thereof, polymeric materials, and composite materials.

12. The support member of claim 3, wherein said cut-out portions comprise a plurality of laser-cut grooves disposed at at least the distal end of said substrate, wherein said grooves are spaced to define a proximal section having a smaller number of grooves per unit of substrate length and a distal section having a greater number of grooves per unit of substrate length, such that said support member has a greater flexibility at said distal end than at said proximal end.

13. The support member of claim 4, wherein said substrate includes grooves disposed along its length from said proximal end to said distal end, and wherein a distance between successive grooves decreases in a gradual and continuous manner from said proximal end to said distal end.

14. The support member of claim 13, wherein at least some of said grooves at said distal end extend into the substrate to a greater depth than the grooves at said proximal end.

15. The support member of claim 13, wherein said support member is formed from stainless steel or a shape memory material.

16. The support member of claim 15, wherein said proximal end of said substrate has a greater diameter than a diameter of said distal end of said substrate.

17. The support member of claim 1, wherein said cut-out portions comprise a plurality of grooves axially disposed along the length of said substrate, said grooves being spaced such that said substrate has a greater number of grooves per unit length of said substrate at said distal end than at said proximal end, at least some of said grooves at said distal end extending deeper into the substrate than the grooves at said proximal end, said substrate having sufficient mass such that the proximal end and the distal end are visible under x-ray fluoroscopy.

18. A method of making a support member for an elongated medical device, comprising:
providing a generally cylindrical substrate having a proximal end and a distal end, and sized to be received in a lumen of the elongated medical device, each of said proximal end and said distal end being visible under medical imagery; and
forming a plurality of cut-out portions along a length of said substrate, said cut-out portions sized and arranged such that the flexibility of said substrate distal end exceeds the flexibility of said proximal end.

19. The method of claim 18, wherein said cut-out portions comprise at least one of circumferential grooves, a spiral-shaped cut-out, and spaced notches.

20. The method of claim 18, wherein said cut-out portions are formed by laser cutting.
